(19) United States
(12) Patent Application Publication
(10) Pub. No.: US 2024/0252778 A1
(43) Pub. Date: Aug. 1, 2024

(54) RESPIRATORY SYSTEM

(71) Applicants: Shivani Rajguru, Bhagagaon (IN); Manas Pratim Borthakur, Guwahati (IN); Habib Md Reazaul Karim, Bongaingaon (IN)

(72) Inventors: Shivani Rajguru, Bhaga Gaon (IN); Manas Pratim Borthakur, Guwahati (IN); Habib Md Reazaul Karim, Bongaingaon (IN); Pradeep Kumar Verma, Ayodhya (IN); Kaushik Choudhury, Gandhinagar (IN)

(21) Appl. No.: 18/605,778

(22) Filed: Mar. 14, 2024

Related U.S. Application Data
(63) Continuation of application No. PCT/IN2022/050821, filed on Sep. 14, 2022.

(30) Foreign Application Priority Data
Sep. 14, 2021 (IN) 202131041443

(51) Int. Cl. A61M 16/20; A61M 16/00; A61M 16/04; A61M 39/10; A61M 39/24

(52) U.S. Cl. CPC A61M 16/208; A61M 16/0003; A61M 16/024; A61M 16/0468; A61M 39/10; A61M 39/24; A61M 2039/1077; A61M 2039/248; A61M 2205/3327; A61M 2205/3334; A61M 2205/50; A61M 2210/065; A61M 2230/40

(57) ABSTRACT

A respiratory system comprising: a tube-within-tube (12, 14), with an outer cylinder tube (14) and an inner cylinder tube (12), with dedicated channels (12a, 14a) for inspired gases (12a) and expired gases (14a) facilitated by valves (2, 3) configured to resist outflow of expired gases, in that, inspired air flows through said inner cylinder tube (12) and expired air flows through said inner cylinder tube (14); a Continuous Expiratory Airway Resistance Valve (CEAR-Valve, Valve 2), being an expiration valve (Valve 2); another unidirectional valve (Valve 3), being an inspiration valve (3).

### RELATED APPLICATIONS

This application claims priority under 35 U.S.C. § 120 to, and is a continuation of, co-pending International Application PCT/IN2022/050821, filed Sep. 14, 2022 and designating the US, which claims priority to IN Application 202131041443, filed Sep. 14, 2021, such IN Application also being claimed priority to under 35 U.S.C. § 119. These IN and International applications are incorporated by reference herein in their entireties.

### FIELD

The invention relates to the field of biomedical engineering. Particularly, this invention relates to lung/breathing related devices. Specifically, this invention relates to a continuous expiratory airway resistance valve.

### BACKGROUND

Incentive Spirometry (IS) is a frequently used visual feedback-based hand-held lung exercise device that helps in lung expansion by facilitating maximum volume respiratory technique. The device is designed to induce sighing or yawning by making the patient take long, slow deep breaths. It is expected to prevent and treat atelectasis (collapse of small lung segments) in alert patients who have a predisposition for shallow breathing. IS leads to the generation of a substantial and sustained increase in the transpulmonary pressure, which leads to expansion of collapsed alveolar units, thereby improving lung function. However, robust evidence to improve the patients' condition is lacking.

Further, the use of the Incentive Spirometry is user-dependent and proper use needs good adherence to instructions; improper use fails to improve the patient's condition. It is also to be noted that it does not impact the positive end-expiratory pressure (PEEP). On the other hand, PEEP is well-known to prevent alveolar collapse and atelectasis.

IS devices, presently available in the market, are manufactured to be used in an awake patient; its mechanism is such that the patient will keep the mouthpiece if the incentive spirometry device is in his/her mouth and inhale the air and then slowly exhale it.

According to prior art, incentive spirometers usually have a vertical cylinder containing a movable piston or ball slidable along with the cylinder. A flexible tube is terminated at one end by a mouthpiece and has its other end connected to an opening in the spirometer that connects with the upper end of the cylinder. When the patient inhales through the mouthpiece it creates a reduced pressure in the tube that is communicated to the upper end of the cylinder. This causes the piston to raise the cylinder/ball. The cylinder is transparent or has a transparent window and is graduated by volume up to its height so that the user can compare the position of the piston against the graduated scale.

### SUMMARY

One of the problems associated with the prior art incentive spirometers is discussed below.

Further, the peri-glottic region is also responsible for maintaining a resistance that forms the basis of physiological PEEP. This physiological PEEP, in turn, prevents the collapse of alveoli and atelectasis. During the process of tracheostomy (creation of a hole in the windpipe through which the patient inspires and expires air), the oral and nasal cavity are bypassed, leading to reduction of the usual resistance to the expired air, which is present in case of mouth breathing. As gas always passes through a path of least resistance, exhaled air escapes through tracheostomy stroma in tracheostomized patients, directly, without being directed towards the nose and mouth. This leads to loss of physiological PEEP. The alveoli collapse leading to atelectasis of the lungs is one of the critical causes of pulmonary complications and failure to liberate the patient from the ventilator or other forms of respiratory support.

In clinical practice, a good number of patients require TT. Classically, tracheostomy, as a procedure, was mainly done in patients with neurological sequelae after Cerebrovascular Accidents (CVA) and as a part of surgeries for large tumors of the head and neck region. However, after the COVID pandemic, the number of patients undergoing tracheostomy for respiratory and lung causes has dramatically increased. Due to the COVID pandemic, a large number of people have developed lung and respiratory complications. In a sub-set of covid recovered patients, post-COVID complications have been seen. These complications mainly affect the lungs and the respiratory system leading to severe compromise of lung capacity and functions. A subset of covid patients become dependent on a ventilator and ultimately need to be tracheostomized.

While non-tracheostomized patients can do incentive spirometry to improve their lung functions and capacities and shown good results, patients on TT, do not have an option to do incentive spirometry as the commercially available IS are not designed to be used in such patients.

One of the other problems associated with the prior art incentive spirometers is discussed below:

Even if some connectors are used to attach the TT and IS, the problem of bypassing the upper airway, which provides physiological resistance and forms the basis of PEEP, is not solved.

During normal physiological breathing, while exhaling, expired air passes through the nose and the mouth and encounters a resistance due to which a positive pressure (physiological positive end-expiratory pressure (PEEP) towards the end of expiration, this helps in keeping the alveoli (small functional units of lungs) in a open state and maintain adequate lung function.

Tracheostomy is a procedure of creating a hole in the trachea (wind pipe) so that air can enter the lungs directly through the windpipe bypassing the oral and nasal cavity. Therefore, in tracheostomized patients as the normal anatomical air passage is bypassed, no resistance is offered to the expired air leading to collapse of the alveoli which is one of the critical causes of pulmonary complications and failure to liberate the patient from the ventilator or other forms of respiratory support. Beneficial effects of PEEP in tracheostomized patients (provided by Mechanical Ventilator)

Normally when a tracheostomized patient is connected to the mechanical ventilator an adjustable level of Extrinsic positive end-expiratory pressure (PEEP) can be provided by the ventilator. The application of positive pressure inside the airways have many beneficial effects: it open or “splint” airways that may otherwise be collapsed; decreasing collapse of small airways (atelectasis); improving oxygen delivery to lungs (alveolar ventilation), and, improve lung function (decreasing VQ mismatch); decrease respiratory effort needed to inflate the lungs (decreases the work of breathing); it has been shown to be helpful in patients in severe COVID pneumonia where the lungs becomes stiff.

Furthermore, the beneficial effects of PEEP can only be provided to tracheostomized patients only till mechanical ventilation is continued. Once mechanical ventilation is weaned off, these effects are lost which leads to collapse of smaller lung units and decrease in lung function. However, the continued application of mechanical ventilator support in tracheostomized patients is not feasible because of following reasons: It dramatically increases health care costs; it has huge financial implications on the patient's family in particular and society in general; Mechanically ventilated patients have higher risks of infections and other complication like decrease oxygen supply (due to equipment failure), need of sedation; Prolong mechanical Ventilation increases length of stay in the intensive care units and also the overall hospital stays; Due to the ongoing pandemic, need for ventilators have gone up drastically and critical equipment and ventilator beds have become a scarce commodity.

One more problem associated with the prior art incentive spirometers is discussed below.

Even if one attaches commercially available valves like PEEP or CPAP (continuous positive airway pressure) valves, the problem is not solved completely. While these valves can provide resistance during expiration or throughout, it increases the work of breathing (effort required to inspire air) breathing by producing resistance to airflow during inspiration.

One additional problem associated with the prior art incentive spirometers is discussed below.

The patients' condition and lung condition are relatively dynamic, and the requirement of resistance and PEEP is variable. There are no currently available valves for IS that can be adjusted to the variable need of resistance.

Currently, commercially available Incentive spirometers are not designed to be used in tracheostomized patients. They are manufactured to be used in awake patients; its mechanism is such that a patient will keep the mouthpiece if the incentive spirometry device is in his/her mouth and inhale air and then slowly exhale it.

An object of the invention is to prevent loss of physiological PEEP. Another object of the invention is to provide incentive spirometry to patients. Yet another object of the invention is to maintain physiological resistance which forms the basis of PEEP during incentive spirometry exercises. Still another object of the invention is to eliminate/reduce resistance to airflow during inspiration whilst doing incentive spirometry exercises. Another object of the invention is to provide a valve for incentive spirometry that can be adjusted to variable needs of resistance.

According to this invention, there is provided a respiratory system comprising:
a tube-within-tube, with an outer cylinder tube and an inner cylinder tube, with dedicated channels for inspired gases and expired gases facilitated by valves configured to resist outflow of expired gases, in that, inspired air flows through the inner cylinder tube and expired air flows through the inner cylinder tube;
a Continuous Expiratory Airway Resistance Valve (CEAR-Valve, Valve 2), being an expiration valve (Valve 2), being a cylindrical co-axial valve configured to be in-line with the outer cylindrical tube and co-axial external to the inner cylindrical tube, the Continuous Expiratory Airway Resistance Valve (CEAR-Valve, Valve 2) being an adjustable spring-loaded resistance unidirectional valve (Valve 2) generating pressure (resistance) to gas flow and maintaining set resistance during time of post-expiratory pause, the Continuous Expiratory Airway Resistance Valve (CEAR-Valve, Valve 2) configured to open only during expiration and configured to close during inspiration, thereby defining a first dedicated channel for expiration, the Continuous Expiratory Airway Resistance Valve (CEAR-Valve, Valve 2) providing an adjustable level of resistance;
another unidirectional valve (Valve 3), being an inspiration valve, stationed at a patient end of the co-axial tube-within-tube inside the inner cylinder tube, the another unidirectional valve (Valve 3) configured to open only during inspiration and configured to close during expiration, thereby defining a second dedicated channel for inspiration; and
entire expired gas, from the Continuous Expiratory Airway Resistance Valve (CEAR-Valve, Valve 2), passing through the outer cylinder encircling the inner cylinder and housing the spring-loaded adjustable valve during expiration.

In at least an embodiment, the Continuous Expiratory Airway Resistance Valve being a unidirectional valve.

In at least an embodiment, an outer cylinder tube ensconces an inner cylinder tube with:
a spaced apart distance between an outer diameter of the inner cylinder tube and an inner diameter of the outer cylinder tube; and
a mouth of the inner cylinder tube abutting a mouth of the outer cylinder tube; and
the inner cylinder tube being co-axial with the outer cylinder tube.

In at least an embodiment, a connector connects a patients' tracheostomy outer end with incentive spirometry (IS), the connector being a universal female adapter.

In at least an embodiment, the valve is configured to instill pressure (resistance) to gas flow ranging from 3 cmH2O to 30 cmH2O.

In at least an embodiment, the inspiration valve (3) is a fish mouth valve for inspiration.

In at least an embodiment, the inspiration valve is a ball valve for inspiration.

### BRIEF DESCRIPTION OF THE ACCOMPANYING DRAWINGS

The patent or application file contains at least one drawing executed in color. Copies of this patent or patent application publication with color drawing(s) will be provided by the Office upon request and payment of the necessary fee.

The invention will now be described in relation to the accompanying drawings, in which:

FIG. 1 illustrates a respiratory system with a Continuous Expiratory Airway Resistance (CEAR) Valve according to this invention;

FIG. 2 illustrates a cross-section of a respiratory system with the Continuous Expiratory Airway Resistance (CEAR) Valve according to this invention;

FIG. 3 illustrates another cross-section of a respiratory system with the Continuous Expiratory Airway Resistance (CEAR) Valve according to this invention;

FIG. 4 illustrates two-dimensional exploded view, of alternative embodiments, of the Continuous Expiratory Airway Resistance (CEAR) Valve according to this invention;

FIG. 5 illustrates three-dimensional exploded view, of alternative embodiments, of the Continuous Expiratory Airway Resistance (CEAR) Valve according to this invention;

FIGS. 6a and 6b illustrates three-dimensional exploded view, of alternative embodiments, of the Continuous Expiratory Airway Resistance (CEAR) Valve according to this invention;

FIGS. 7a, 7b, and 7c illustrate various views of the valve configuration (12, 14, CEAR) used in this invention;

FIG. 8 illustrates air flow property of system at inlet flow rate—5 m/s;

FIG. 9 illustrates pressure at 30 WC at exhalation presses;

FIG. 10 illustrates FEA model of used spring;

FIG. 11 illustrates air flow at 5 mm spring deflection and 1.08 n pressure on plate;

FIG. 12 illustrates results with valve design at 30 wc pressure such that it is safe to handle pressure and structural strength is safe. This structure is qualified to given result;

FIG. 13 illustrates a sectional view of the respiratory system, with sensors, of this invention; and

FIG. 14a and FIG. 14b also illustrate sectional views of the respiratory system, with sensors, of this invention.

### DETAILED DESCRIPTION

According to this invention, there is provided a respiratory system.

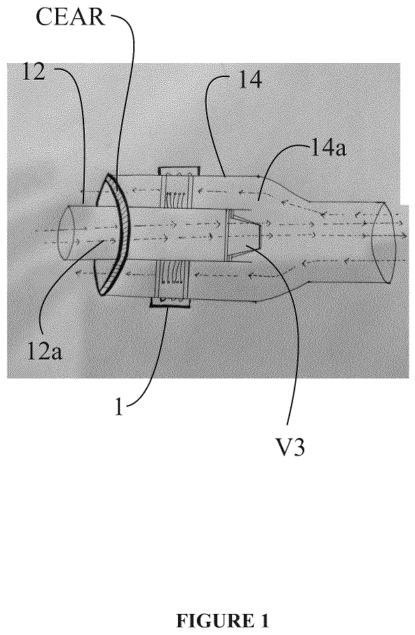

FIG. 1 illustrates a respiratory system with a Continuous Expiratory Airway Resistance (CEAR) Valve according to this invention.

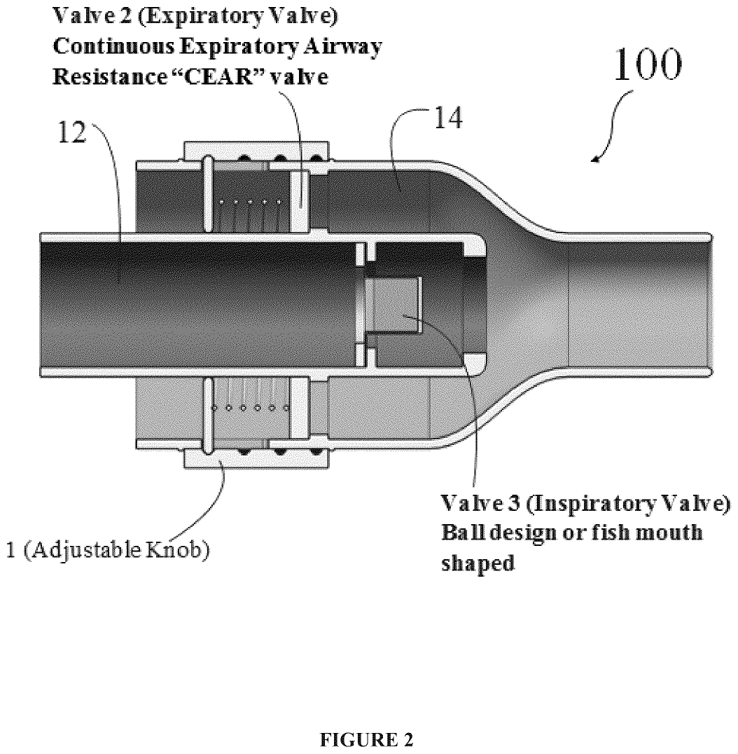

FIG. 2 illustrates a cross-section of a respiratory system with the Continuous Expiratory Airway Resistance (CEAR) Valve according to this invention.

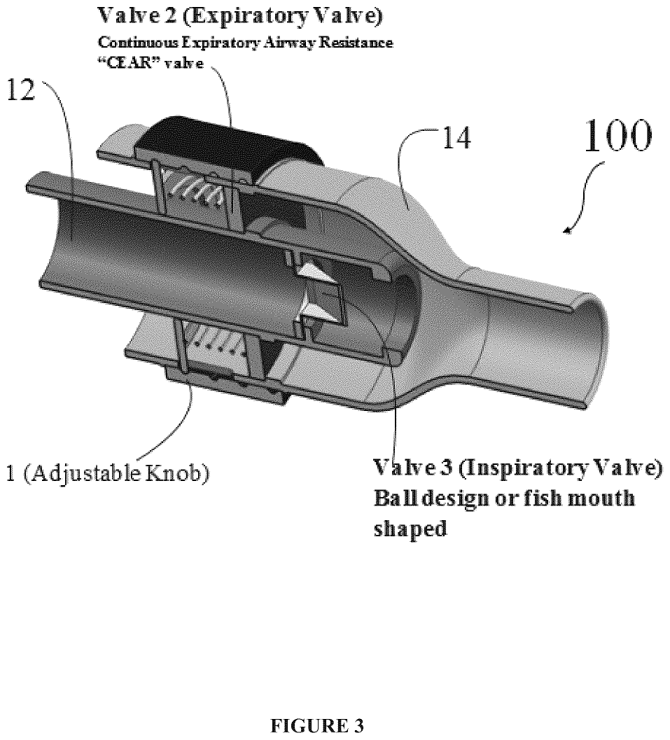

FIG. 3 illustrates another cross-section of a respiratory system with the Continuous Expiratory Airway Resistance (CEAR) Valve according to this invention.

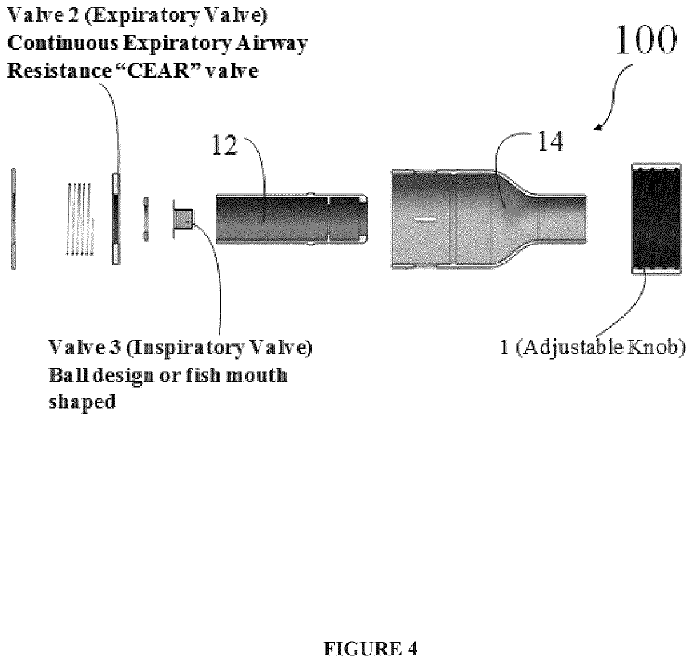

FIG. 4 illustrates two-dimensional exploded view, of alternative embodiments, of the Continuous Expiratory Airway Resistance (CEAR) Valve according to this invention.

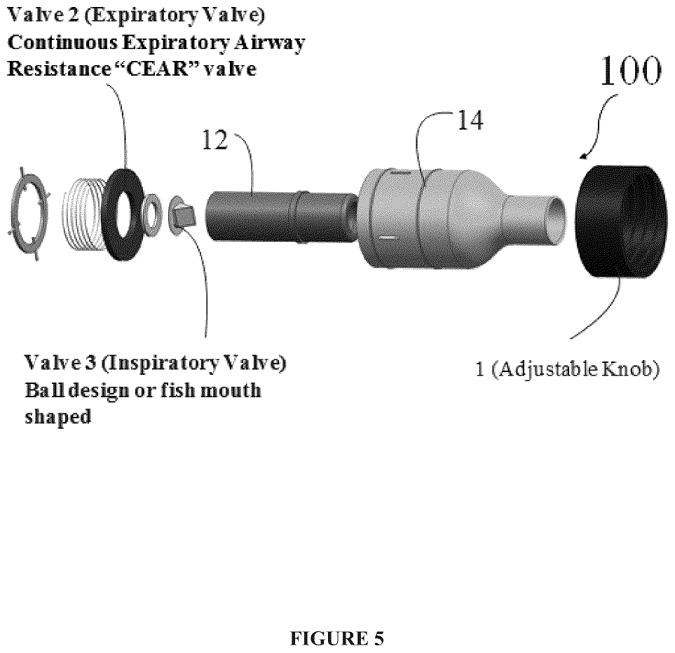

FIG. 5 illustrates three-dimensional exploded view, of alternative embodiments, of the Continuous Expiratory Airway Resistance (CEAR) Valve according to this invention.

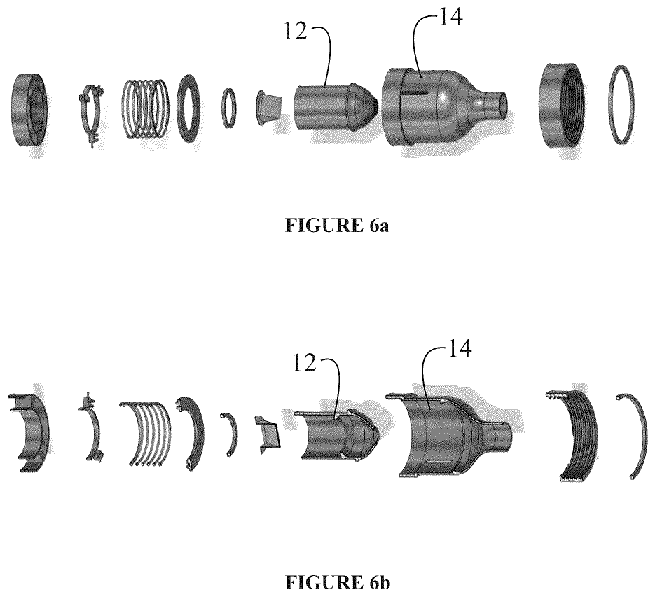

FIGS. 6a and 6b illustrates three-dimensional exploded view, of alternative embodiments, of the Continuous Expiratory Airway Resistance (CEAR) Valve according to this invention.

Typically, the valve/device, of this invention, is intended to be used along with pre-existing devices called Incentive spirometers (IS) and Tracheostomy tubes (TT). This valve broadens the scope of use of commercially available incentive spirometers in tracheostomized patients.

In at least an embodiment, of this invention, the CEAR valve comprises a tube-within-tube (12, 14) design with dedicated channels for inspired and expired gases facilitated by valves (2, 3); this resists outflow of expired gases. However, there is none or negligible resistance to inflow of gas (inspiration) so that there is no increase of work of breathing for the patient.

In preferred embodiments, the tube size is a universal 15 mm female connector at the connecting end to be easily connected to the TT. The size and weight is minimal.

In preferred embodiments, resistance is adjustable from 3 cmm H2O to 30 cmH2O.

In at least an embodiment, of this invention, the Continuous Expiratory Airway Resistance Valve (CEAR-Valve) is a cylindrical co-axial design and placed in a limb (breathing line) connecting a patients' tracheostomy outer end with incentive spirometry (IS). The connection is a universal female adapter (preferably, 15 mm) to get attached to the tracheostomy tube. The breathing line (tube) houses the ‘Continuous Expiratory Airway Resistance Valve’ (CEAR-Valve) which is an adjustable spring-loaded resistance valve (Valve 2 in the figures). The CEAR-Valve (Valve 2 in the figures) is a spring-based pressure valve generating a minimum of 3 cmH2O pressure (resistance) to gas flow. It provides resistance only during expiration and maintains set resistance during time of post-expiratory pause. However, the valve can be one delivering a variable and adjustable resistance. The valve allows only unidirectional flow. It does not allow air entry during inspiration through the incentive spirometry (IS) but only allows expiration against a set resistance (can be set by the user or clinician as deemed necessary).

In at least an embodiment, one more unidirectional valve (Valve 3 in the figures), is stationed at the patient end of the co-axial cylindrical device inside the inner cylinder. This valve 3 opens only during inspiration and closes during expiration. The internal diameter of the tube housing this valve, preferably, is more than 10 mm. Therefore, the patient inspires without much resistance. On the other hand, the entire expired gas passes through the outer channel encircling the inner tube and housing the spring-loaded adjustable valve during expiration. The design of the Valve 2 and Valve 3 is as shown in Figures. The inner cylinder, of the co-axial system, is deficient towards the patient (TT) end so that the outer cylinder fits the tracheostomy tube snugly.
Reference numeral 1: adjustable knob
Reference numeral Valve 2: expiration valve
Reference numeral Valve 3: inspiration valve

In at least an embodiment, an outer cylinder tube (14) ensconces an inner cylinder tube (12) with:
a spaced apart distance between an outer diameter of the inner cylinder tube (12) and an inner diameter of the outer cylinder tube (14); and
a mouth of the inner cylinder tube (12) abutting a mouth of the outer cylinder tube (14); and
the inner cylinder tube (12) being co-axial with the outer cylinder tube (14).

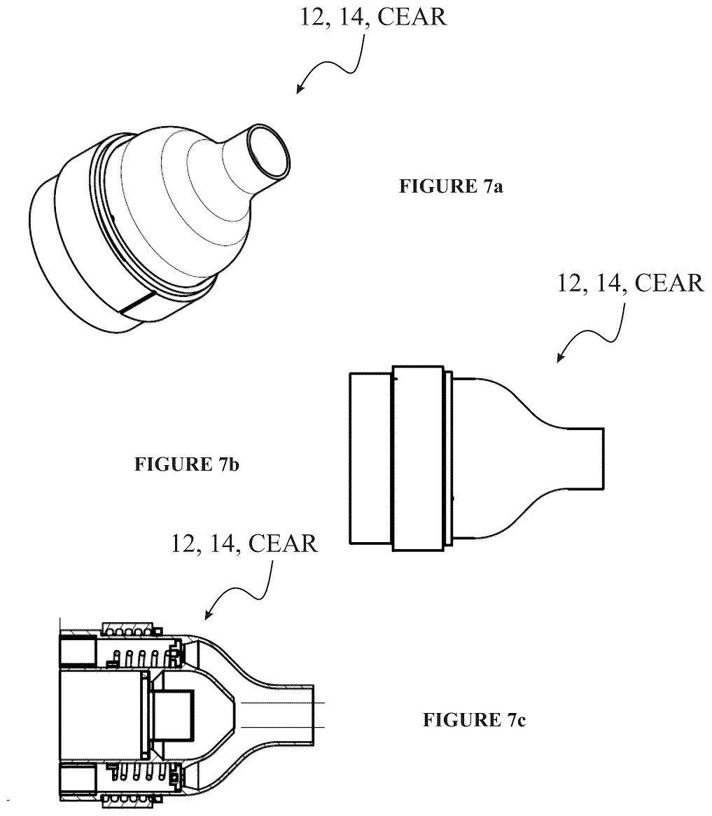

FIGS. 7a, 7b, and 7c illustrate various views of the valve configuration (12, 14, CEAR) used in this invention.

In typical embodiments, the inspiration valve (3) is a fish mouth valve for inspiration; this valve is useful since it does not reduce the effective diameter of the inspiratory limb for gas flow and it does not increase resistance.

In alternative embodiments, the inspiration valve (3) is a ball valve for inspiration.

In at least an embodiment, the main feature of the CEAR valve, of this invention, is that it enables tracheostomized patients to use incentive spirometry devices. This device provides a desired yet adjustable/selectable resistance to outflow of expired gases, which work like PEEP; a ‘critical’ factor to keep the alveoli inflated. It is, in turn, likely to help a patient in: Lung exercise; Prevent lung atelectasis; Reduce the postoperative pulmonary complication; Improve the oxygenation by improving the ventilation-perfusion mismatch.

The current invention seeks to create/establish a PoC for a device which is a Continuous Expiratory Airway Resistance “CEAR” valve device that can be used on Tracheostomized patients and yield the following benefits:
Provision of Positive pressure (PEEP) and thus its benefits, as stated above, to tracheostomized patients without the need of mechanical ventilator.
Improve Lung function and facilitate early liberation of tracheostomized patients from mechanical ventilator.
Decrease complications associated with prolonged mechanical ventilation.
Decrease the length of stay in the intensive care units (due to early weaning).
Decrease health care costs decrease the financial burden on the patient's family and the society as a whole.
‘CEAR’ will enable the use of commercially available Incentive Spirometry devices in tracheostomized patients and thus provide its benefits to them.
‘CEAR’ valve is a market creating innovation which will unfold a large unmet need. It will increase the market size for incentive spirometry devices by enabling their use in tracheostomized patients.

This invention's valve broadens the scope of use of commercially available incentive spirometers in tracheostomized patients. One can broaden the applicability of the device by not limiting its use only as a connector for IS. One can also add that this device might have applicability in anaesthesia circuits (self-inflating manual resuscitator bags, bain's circuits, ventilators, and ventilator circuits, etc.) and for providing PEEP support to other devices which can be connected to a tracheostomy tube or Endotracheal tube). With subsequent modifications, this device would also be used for bed side measurement of lung functions like Peak Expiratory flow rate (PEFR) and Forced Expiratory Volume in 1st second FEV1 etc.

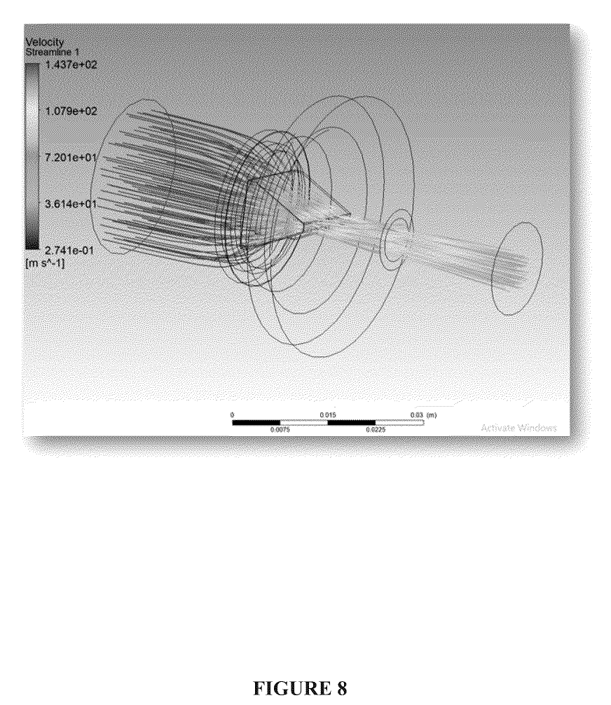

FIG. 8 illustrates air flow property of system at inlet flow rate—5 m/s.

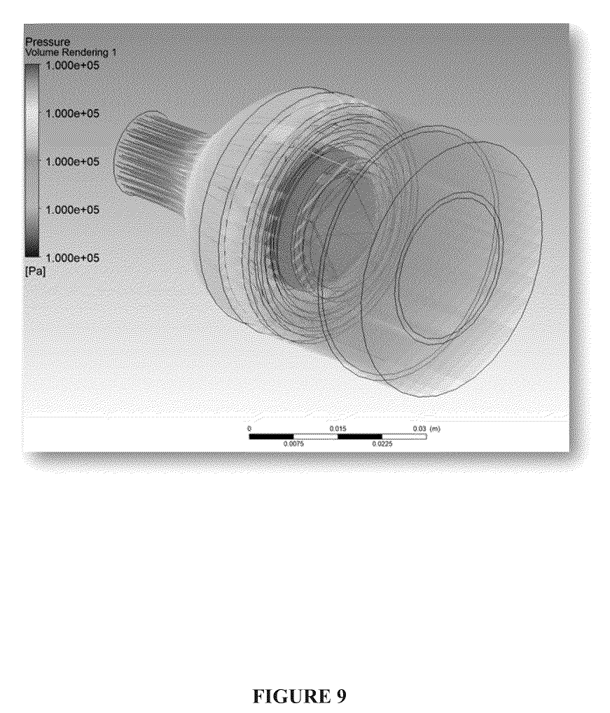

FIG. 9 illustrates pressure at 30 WC at exhalation presses. This creates pressure on spring plate. Load is 1.08 N. Inlet air—5 M/s.

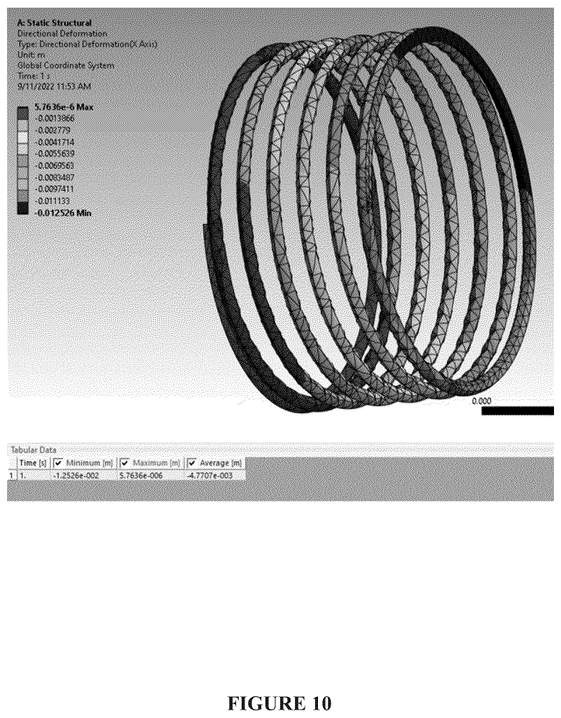

FIG. 10 illustrates FEA model of used spring. APPLY LOAD—1.08 N. SPRING DEFLECTION—5.2 mm.

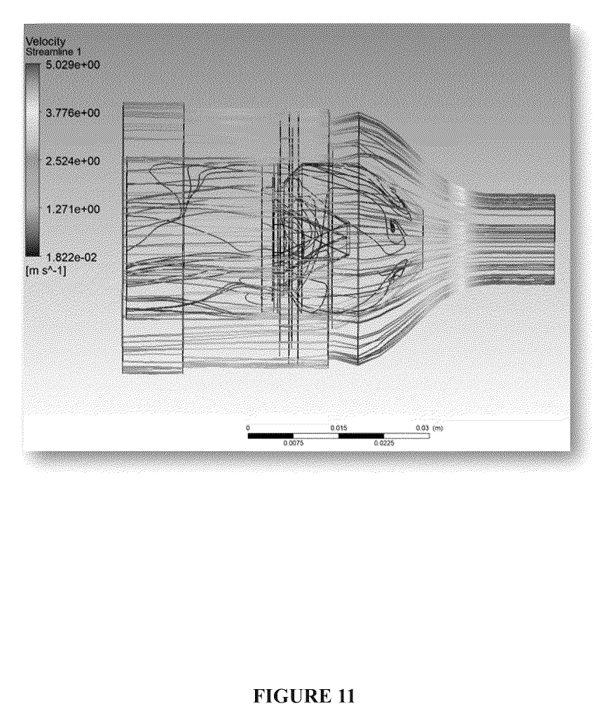

FIG. 11 illustrates: Air flow at 5 mm spring deflection and 1.08 n pressure on plate. Air flow 5 m/s.

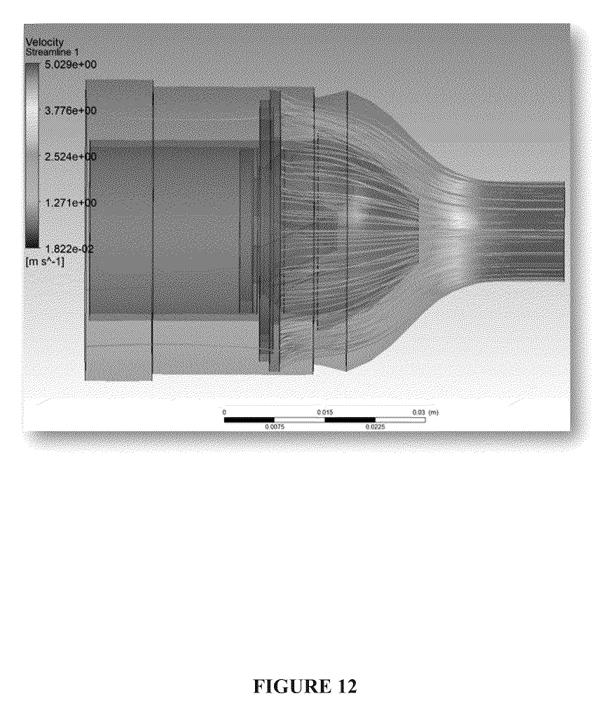

FIG. 12 illustrates results with valve design at 30 wc pressure such that it is safe to handle pressure and structural strength is safe. This structure is qualified to given result

Table 1 illustrates readings from a CFD model showing the pressure at spring plate is 1.08 N at 30 wc applying inlet air value through exhalation Process.

TABLE 1

| | SPRING | WC |
|---|---|---|
| SN | SETTING | PRESSURE |
| 1 | 1 mm | 6 |
| 2 | 2 mm | 12 |
| 3 | 3 mm | 18 |
| 4 | 4 mm | 24 |
| 5 | 5 mm | 30 |

It can be seen that at 5 mm spring setting, the value of force become equal to the 30 wc pressure. So, if one compresses spring to 1 mm, the wc pressure is 6 wc. The inlet air flow through inhale (30 lpm, 60 lpm, 90 lpm) does not affect the pressure inside tube—because one side valve should be closed.

The respiratory system, of this invention, has a dedicated inspiratory channel (12) and an expiratory channel (14). When a person uses an incentive spirometer (respiratory system), they take in a deep breath and a piston, in the incentive spirometer, moves and provides a visual clue about the Tidal Volume generated by the person which incentives the person to perform better in their next breath.

In accordance with this invention, as explained below, a person blows into a mouth piece and the spirometer (respiratory system) calculates desired value through different sensors. In the CEAR Respiratory system, of this invention, Pressure sensors and Differential Pressure sensors are integrated into the expiratory channel (14) so that an objective assessment of lung function can done in patients.

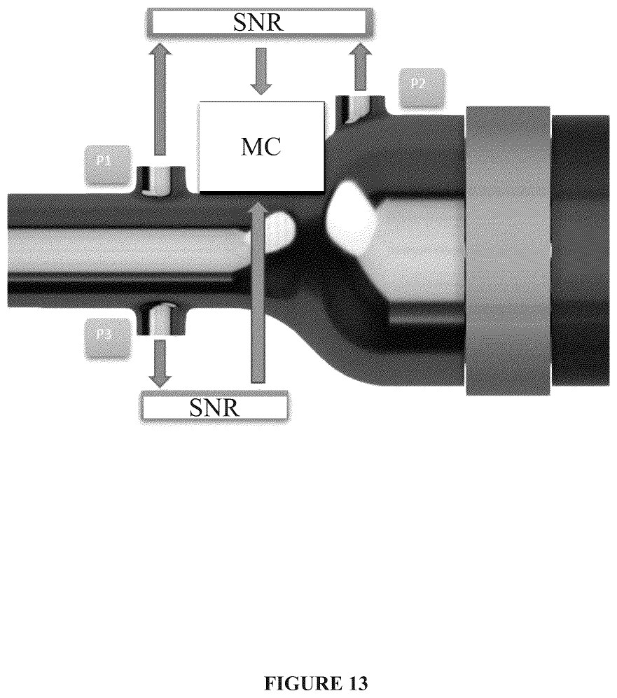

FIG. 13 illustrates a sectional view of the respiratory system, with sensors, of this invention.

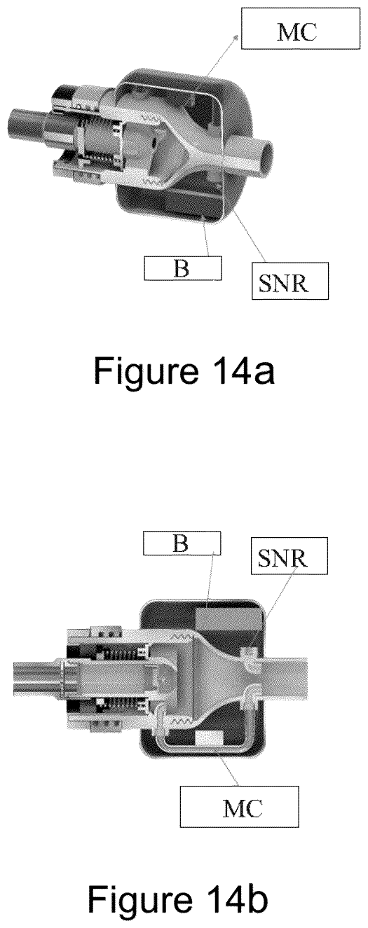

FIG. 14a and FIG. 14b also illustrate sectional views of the respiratory system, with sensors, of this invention.

In at least an embodiment, one or more differential air pressure sensors (DSNR) are employed between a first port (P1) and a second port (P2). P1 is the proximal port of the differential pressure sensor closer to the patient's tracheostomy tube or mouth piece (depending on whether used for tracheostomized patients or normal patients without tracheostomy). P2 is the distal port of the differential which pressure sensor placed approximately 1-1.5 cm from the proximal port P1 created by which measures the two different readings of pressure created by created by flow of air during exhalation. Differential pressure reading, from the differential air pressure sensor converted into corresponding flow rate by a microcontroller (MC) using method of polynomial curve fitting on differential pressure (Pascal) vs standard flow rate measurements (LPM).

In at least an embodiment, one or more air pressure sensors (SNR) measures gauge pressure at a third port (P3) (in cmH2O). P3 is an independent port placed at the level of port P1 but at the opposite side of the expiratory limb as shown in the figure inside the chamber during exhalation. Reference numeral B refers to battery pack. Readings from the sensors (DSNR, SNR), correlative to the ports (P1, P2, P3) is given to a microcontroller (MC) in order to monitor the flow rate measurements (LPM) and the gauge pressure (cmH2O) readings in real time.

This invention is a unique portable spirometer such that that apart from providing objective evaluation of lung function (diagnostic) it also provides adjustable but continuous positive pressure which has therapeutic benefit of improving lung function by opening up collapsed parts of the smaller airways (thus, providing diagnostic as well as therapeutic benefit at the same time. It has specific use in tracheostomy patients. It can also be used for non-tracheostomized patients breathing normally by using a mouth piece connected to the patient end of the device. In non-tracheostomized patients also the device will provide both diagnostic and therapeutic benefits.

The technical advantages of the current invention, are as follows: When CEAR valve, of this invention, is connected to IS; IS can be used in tracheostomized patients. It can also be used for non-tracheostomized patients breathing normally by using a mouth piece connected to the patient end of the device. In non-tracheostomized patients also the device will provide both diagnostic and therapeutic benefits; CEAR valve, of this invention, prevents loss of lung recruitment and prevents the collapse of the smaller lung units (alveoli) and prevent lung function in the tracheostomized patient (by proving PEEP); It is likely to decrease pulmonary complications in tracheostomized patients; It is likely to facilitate patient recovery; It is likely to decrease the length of stay in intensive care or ward, which will decrease overall health costs.

The TECHNICAL ADVANCEMENT of this invention lies in providing a respiratory system with Continuous Expiratory Airway Resistance Valve (CEAR valve) which is designed such that it provides a predetermined resistance to outflow of expired gases and mimics normal physiology (resistance to expired air provided by the structures present in the nose and mouth) during normal breathing through nose and mouth. Further, it maintains positive end-expiratory pressure (PEEP) to keep alveoli, of a patient, open after expiration, which is usually maintained by physiological PEEP, and physiological PEEP is lost to some extent in patients with TT. The valve is designed such that it provides resistance selectively to the expiration as the set resistance will persist during the end-expiratory pause period mimicking PEEP. The tube within tube design will have a dedicated inspiratory channel which provides very low or negligible resistance to the inspiration. The CEAR valve has the facility to adjust the resistance during expiration, and it will range between 3-20 cmH2O.

While this detailed description has disclosed certain specific embodiments for illustrative purposes, various modifications will be apparent to those skilled in the art which do not constitute departures from the spirit and scope of the invention as defined in the following claims, and it is to be distinctly understood that the foregoing descriptive matter is to be interpreted merely as illustrative of the invention and not as a limitation.

1. A respiratory system comprising:
a tube-within-tube having an outer cylinder tube for expired air flow and an inner cylinder tube for inspired air flow and valves configured to resist outflow of expired gases;
a continuous expiratory airway resistance valve that is cylindrical and co-axial with the outer cylindrical tube and the inner cylindrical tube, wherein the continuous expiratory airway resistance valve is an adjustable, spring-loaded, resistance unidirectional valve configured to generate pressure to gas flow and maintain a set resistance during time of post-expiratory pause and configured to open only during expiration and to close during inspiration so as to provide a first dedicated channel for expiration, and wherein the continuous expiratory airway resistance valve provides an adjustable level of resistance;
an inspiration valve that is a unidirectional valve, wherein the inspiration valve is located at a patient end of the tube-within-tube and inside the inner cylinder tube, wherein the inspiration valve is configured to open only during inspiration and to close during expiration so as to provide a second dedicated channel for inspiration, and wherein the continuous expiratory airway resistance valve is configured such that all the expired gas passes through only the outer cylinder tube housing the spring-loaded adjustable valve during expiration.

2. The system of claim 1, wherein the continuous expiratory airway resistance valve is a unidirectional valve.

3. The system of claim 1, wherein the outer cylinder tube is spaced apart from the inner cylinder tube, wherein a mouth of the inner cylinder tube abuts a mouth of the outer cylinder tube, and wherein the inner cylinder tube is co-axial with the outer cylinder tube.

4. The system of claim 1, further comprising:
a connector configured to connect to a patients tracheostomy outer end with incentive spirometry, wherein the connector is a universal female adapter.

5. The system of claim 1, wherein the continuous expiratory airway resistance valve is configured to instill pressure to the gas flow ranging from 3 cm H2O to 30 cm H2O.

6. The system of claim 1, wherein the inspiration valve is a fish mouth valve for inspiration.

7. The system of claim 1, wherein the inspiration valve is a ball valve for inspiration.

8. The system of claim 1, further comprising:
a differential air pressure sensor between a first port and a second port, wherein the first port is a proximal port of the differential air pressure sensor, wherein the second port is a distal port of the differential air pressure sensor spaced apart from the proximal port, wherein the differential air pressure sensor is configured to measure two different readings of pressure created by created by flow of air during exhalation;
an air pressure sensor configured to measure gauge pressure at a third port at a level of the first port; and
a microcontroller configured to receive data from the differential air pressure sensor and the air pressure sensor to monitor the flow rate measurements and the gauge pressure readings in real time.
